(19) United States
(12) Patent Application Publication
(10) Pub. No.: US 2020/0113545 A1
(43) Pub. Date: Apr. 16, 2020

(54) DIAGNOSTIC ASSISTANCE METHOD

(71) Applicant: Acer Incorporated, New Taipei City (TW)

(72) Inventors: Jyun-Guo Wang, New Taipei City (TW); Yin-Hsong Hsu, New Taipei City (TW)

(73) Assignee: Acer Incorporated, New Taipei City (TW)

(21) Appl. No.: 16/504,619

(22) Filed: Jul. 8, 2019

(30) Foreign Application Priority Data
Oct. 16, 2018 (TW) 107136315

(51) Int. Cl. A61B 8/08; A61B 7/04; A61B 8/00; A61B 5/00; G06N 3/08; G06N 20/20

(52) U.S. Cl. CPC A61B 8/5223; A61B 7/04; G06N 20/20; A61B 5/7267; G06N 3/08; A61B 8/463

(57) ABSTRACT

A diagnostic assistance method is provided in the invention. The diagnostic assistance method includes the steps of generating audio data with a stethoscope; generating image data via an ultrasonic device; processing the audio data through a first processing module to generate a first result; processing the image data through a second processing module to generate a second result; and generating a diagnostic assistance result according to the first result and the second result.

### CROSS REFERENCE TO RELATED APPLICATIONS

This Application claims priority of TW Patent Application No. 107136315 filed on Oct. 16, 2018, the entirety of which is incorporated by reference herein.

### BACKGROUND OF THE INVENTION

### Field of the Invention

The invention generally relates to diagnostic assistance technology, and more particularly, to diagnostic assistance technology for generating a diagnostic assistance result according to audio data generated by a stethoscope and image data generated by an ultrasonic device.

### Description of the Related Art

In traditional medical treatment, stethoscopes are used independently of ultrasonic devices. In other words, they are not used at the same time. Therefore, the accuracy of some diagnoses may suffer.

However, the results obtained from a stethoscope and those from an ultrasonic device are correlative. For example, the stethoscope can be used to hear the disease syndrome, but it cannot be used to confirm the location of the disease syndrome accurately. However, the ultrasonic device can provide the location of the disease syndrome. On the other hand, the ultrasonic device can be used to obtain the location of the disease syndrome accurately, but it is more difficult to recognize the disease syndrome according to the image generated by the ultrasonic device than it is to recognize the disease syndrome using a stethoscope. Therefore, the accuracy of the results obtained from an ultrasonic device may be decreased.

Therefore, if the advantages of the stethoscope and the ultrasonic device can be combined, the accuracy of the diagnosis will improve.

### BRIEF SUMMARY OF THE INVENTION

A diagnostic assistance method for generating a diagnostic assistance result according to audio data generated by a stethoscope and image data generated by an ultrasonic device is provided to overcome the problems mentioned above.

An embodiment of the invention provides a diagnostic assistance method. The diagnostic assistance method comprises the steps of generating audio data with a stethoscope; generating image data via an ultrasonic device; processing the audio data to generate a first result by a first processing module; processing the image data to generate a second result by a second processing module; and generating a diagnostic assistance result according to the first result and the second result.

According some embodiments of the invention, the first processing module processes the audio data through a first algorithm to generate the first result, and the second processing module processes the image data through a second algorithm to generate the second result.

According some embodiments of the invention, the diagnostic assistance method comprises the step of analyzing the first result and the second result through a third algorithm by a third processing module to generate the diagnostic assistance result.

Other aspects and features of the invention will become apparent to those with ordinary skill in the art upon review of the following descriptions of specific embodiments of diagnostic assistance method.

### BRIEF DESCRIPTION OF THE DRAWINGS

The invention will become more fully understood by referring to the following detailed description with reference to the accompanying drawings, wherein:

FIG. 1 is a block diagram of a diagnostic assistance system 100 according to an embodiment of the invention; and

FIG. 2 is a flow chart 200 illustrating a diagnostic assistance method according to an embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

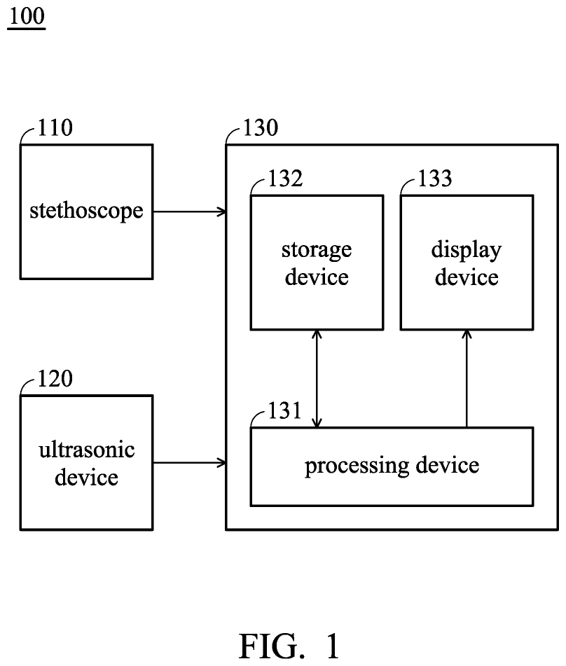

FIG. 1 is a block diagram of a diagnostic assistance system 100 according to an embodiment of the invention. As shown in FIG. 1, the diagnostic assistance system 100 may comprise a stethoscope 110, an ultrasonic device 120 and a diagnostic assistance device 130. FIG. 1 presents a simplified block diagram in which only the elements relevant to the invention are shown. However, the invention should not be limited to what is shown in FIG. 1.

As shown in FIG. 1, in the embodiments of the invention, the diagnostic assistance device 130 may comprise a processing device 131, a storage device 132 and a display device 133. According to the embodiments of the invention, the diagnostic assistance device 130 may be a smart phone, a tablet, a desk computer or a notebook, but the invention should not be limited to thereto. In addition, it should be noted that the diagnostic assistance device 130 is only used to illustrate the embodiments of the invention, but the invention should not be limited to thereto. The diagnostic assistance device 130 also can comprise other elements.

According to an embodiment of the invention, the stethoscope 110 may be a digital stethoscope. The stethoscope 110 may be configured to obtain the audio data (or audio signal) corresponding to human organs (e.g. heart, lung, intestines and stomach). After the stethoscope 110 obtains the audio data, the stethoscope 110 may transmit the obtained audio data to the diagnostic assistance device 130 through a wire or a wireless communication. According to an embodiment of the invention, the audio data generated by the stethoscope 110 may be temporarily stored in the storage device 132 of the diagnostic assistance device 130.

According to an embodiment of the invention, the ultrasonic device 120 may be an ultrasonic probe. The ultrasonic device 120 comprises a transmitter and a receiver (not shown in figures). The transmitter of the ultrasonic device 120 may transform the electrical signal into a sound wave signal (i.e. ultrasonic signal), and transmit the sound wave signal to the human body. The receiver of the ultrasonic device 120 may receive the sound wave signal reflected from the human body, and transform the sound wave signal into an electrical signal. Then, the receiver of the ultrasonic device 120 may transform the electrical signal into a 2-dimensional (2D) image (i.e. image data). After the ultrasonic device 120 obtains the image data corresponding to human organs, the ultrasonic device 120 may transmit the image data corresponding to human organs to the diagnostic assistance device 130 through a wire or a wireless communication. According to an embodiment of the invention, the image data generated by the ultrasonic device 120 may be temporarily stored in the storage device 132 of the diagnostic assistance device 130.

According to an embodiment of the invention, the storage device 132 may be a volatile memory device, e.g. a dynamic random access memory (DRAM), but the invention should not be limited thereto. According to another embodiment of the invention, the storage device 132 may be a nonvolatile memory device, e.g. a flash memory or, a read only memory (ROM) but the invention should not be limited thereto. According to an embodiment of the invention, the storage device 132 may store the software codes, firmware codes, training audio data, training image data, and so on. According to the embodiments of the invention, the training audio data means the audio data which is suspected of suffering from the disease and marked by the doctor previously. For example, during the prior diagnostic process for the disease syndromes of different organs, the doctor may mark the audio waveform which is suspected of suffering from the disease in the audio data, and the marked audio data may be stored in the storage device 132 to be taken as the training audio data. In addition, in the embodiments of the invention, the training image data means the image data which is suspected of suffering from the disease and marked by the doctor previously. For example, during the prior diagnostic process for the disease syndromes of different organs, the doctor may mark the image feature which is suspected of suffering from the disease in the image data, and the marked image data may be stored in the storage device 132 to be taken as the training image data.

According to an embodiment of the invention, after the diagnostic assistance device 130 obtains the audio data and image data from the stethoscope 110 and the ultrasonic device 120 respectively, a first processing module (not shown in figure) of the processing device 131 of the diagnostic assistance device 130 may obtain the training audio data and the audio data generated by the stethoscope 110 from the storage device 132, and the first processing module may process and analyze the audio data generated by the stethoscope 110 according to the training audio data and the audio data generated by the stethoscope 110 to generate a first result. Specifically, the processing device 131 may compare the training audio data with the audio data generated by the stethoscope 110 to determine which part of the audio data generated by the stethoscope 110 may be suspected of suffering from the disease, and the processing device 131 may mark the part of the audio data which may be suspected of suffering from the disease to generate the first result.

In addition, a second processing module (not shown in figure) of the processing device 131 of the diagnostic assistance device 130 may obtain the training image data and the image data generated by the ultrasonic device 120 from the storage device 132, and the second processing module may process and analyze the audio data generated by the ultrasonic device 120 according to the training image data and the image data generated by the ultrasonic device 120 to generate a second result. Specifically, the processing device 131 may compare the training image data with the image data generated by the ultrasonic device 120 to determine which part of the audio data generated by the ultrasonic device 120 may be suspected of suffering from the disease, and the processing device 131 may mark the part of the image data which may be suspected of suffering from the disease to generate the second result.

According to an embodiment of the invention, the first processing module of the processing device 131 may process and analyze the audio data generated by the stethoscope 110 through a first algorithm, and the second processing module of the processing device 131 may process and analyze the audio data generated by the ultrasonic device 120 through a second algorithm. According to an embodiment of the invention, the first algorithm may be a Recurrent Neural Network (RNN) deep learning algorithm and the second algorithm may be a Convolutional Neural Network (CNN) deep learning algorithm, but the invention should not be limited thereto. According to some embodiments of the invention, the first algorithm also may be the CNN deep learning algorithm or another deep learning algorithm, and the second algorithm also may be the RNN deep learning algorithm or another deep learning algorithm. According to some embodiments of the invention, the first algorithm and the second algorithm may be the combination of two deep learning algorithms, e.g. the combination of the CNN deep learning algorithm and the RNN deep learning algorithm, but the invention should not be limited thereto.

In the RNN deep learning algorithm, each element of a sequence may be performed the same operation through the back propagation and memory mechanism according to the information in the sequence. Further, in the RNN deep learning algorithm, the current output may be affected by the prior output. The first processing module of the processing device 131 may adopt the RNN deep learning algorithm to compare the training audio data with the audio data generated by the stethoscope 110 to generate the first result.

The structure of the CNN deep learning algorithm may comprise a Convolution Layer, a Pooling Layer, and a Fully Connected Layer. In the Convolution Layer, the convolution calculation may be performed for the image and the dedicated feature detector to extract the features in the image. In the Pooling Layer, a pooling method (e.g. Max Pooling method, but the invention should not be limited thereto) may be adopted to divide the image processed by the Convolution Layer into a plurality of blocks, and select the maximum value of each block from each block. In the Fully Connected Layer, the results from the Pooling Layer may be flattened. In addition, the CNN deep learning algorithm may have many types, such as a region CNN (R-CNN), a fast R-CNN, and a faster R-CNN. The second processing module of the processing device 131 may adopt the CNN deep learning algorithm to compare the training image data with the image data generated by the ultrasonic device 120 to generate the second result.

According to an embodiment of the invention, the user can adjust the parameters (e.g. number of epoch, learning rate, objective function, weight initialization and regularization, but the invention should not be limited thereto) of the RNN deep learning algorithm and the CNN deep learning algorithm according to the first result and the second result.

According to an embodiment of the invention, after the first result and the second result are generated, a third processing module (not shown in figures) of the processing device 131 may receive the first result and the second result, and generate a diagnostic assistance result according to the first result and the second result. According to an embodiment of the invention, the third processing module of the processing device 131 may analyze the first result and the second result through a third algorithm to generate the diagnostic assistance result. According to an embodiment of the invention, the third algorithm may be an Ensemble Learning algorithm, but the invention should not be limited thereto. In the Ensemble Learning algorithm, the prediction results (e.g. the first result and the second result) of different classifiers may be considered comprehensively, and different weights may be provided to different prediction results to obtain better prediction result (i.e. diagnostic assistance result).

After the processing device 131 generates the diagnostic assistance result, the processing device 131 may output the diagnostic assistance result to the display device 133. After the display device 133 receives the diagnostic assistance result, the display device 133 may display the diagnostic assistance result for doctor's reference. According to an embodiment of the invention, the diagnostic assistance result may be marked audio data, marked image data, or text data, but the invention should not be limited thereto. For example, if the diagnostic assistance result is text data, the diagnostic assistance result may comprise the description of the disease syndromes of the human body, e.g. the possible location of the disease syndrome or the probability of the disease syndrome, but the invention should not be limited thereto.

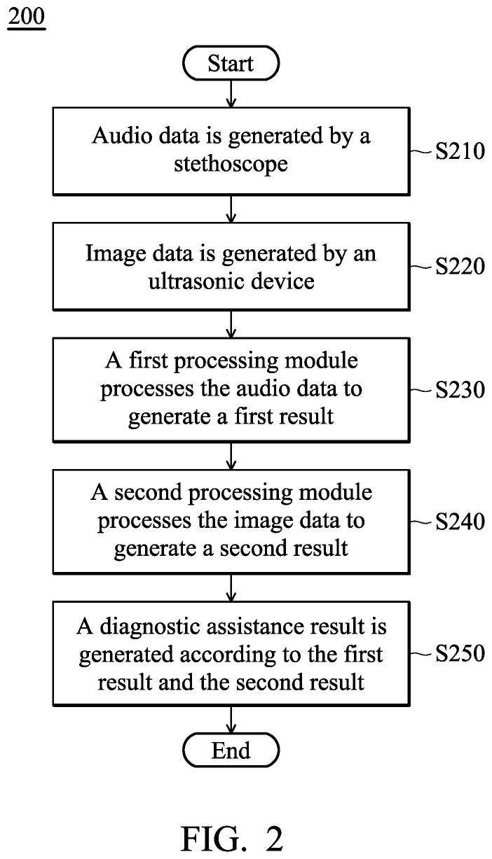

FIG. 2 is a flow chart 200 illustrating a diagnostic assistance method according to an embodiment of the invention. The diagnostic assistance method can be applied to the diagnostic assistance system 100. In step S210, audio data is generated by a stethoscope of the diagnostic assistance system 100. In step S220, image data is generated by an ultrasonic device of the diagnostic assistance system 100. In step S230, a first processing module of the diagnostic assistance device of the diagnostic assistance system 100 processes the audio data generated by the stethoscope to generate a first result. In step S240, a second processing module of the diagnostic assistance device of the diagnostic assistance system 100 processes the image data generated by the ultrasonic device to generate a second result. In step S250, the diagnostic assistance device of the diagnostic assistance system 100 generates a diagnostic assistance result according to the first result and the second result.

According to an embodiment of the invention, in the diagnostic assistance method, the first processing module processes the audio data generated by the stethoscope through a first algorithm to generate the first result, and the second processing module processes the image data generated by the ultrasonic device through a second algorithm to generate the second result. According to an embodiment of the invention, the first algorithm may be a Recurrent Neural Network (RNN) deep learning algorithm and the second algorithm may be a Convolutional Neural Network (CNN) deep learning algorithm. According to an embodiment of the invention, in the diagnostic assistance method, the first processing module may compare the training audio data with the audio data generated by the stethoscope through the first algorithm to generate the first result, and the second processing module may compare the training image data with the image data generated by the ultrasonic device through the second algorithm to generate the second result.

According to an embodiment of the invention, the diagnostic assistance method further comprises a third processing module of the diagnostic assistance device of the diagnostic assistance system 100 analyzing the first result and the second result through a third algorithm to generate the diagnostic assistance result. According to an embodiment of the invention, the third algorithm may be an Ensemble Learning algorithm.

According to an embodiment of the invention, the diagnostic assistance method further comprises a display device of the diagnostic assistance system 100 displaying the diagnostic assistance result. According to an embodiment of the invention, the diagnostic assistance result may be marked audio data, marked image data, or text data, but the invention should not be limited thereto.

According to the diagnostic assistance method provided in the embodiments of the invention, the results from the stethoscope and the results from the ultrasonic device can be integrated and the deep learning algorithms can enhance the correlation between the results obtained by the stethoscope and the results obtained by the ultrasonic device. Therefore, the diagnostic assistance method provided in the embodiments of the invention can provide accurate and effective diagnostic assistance results for a doctor's reference.

Reference throughout this specification to “one embodiment” or “an embodiment” means that a particular feature, structure, or characteristic described in connection with the embodiment is included in at least one embodiment of the invention, but do not denote that they are present in every embodiment. Thus, the appearances of the phrases “in one embodiment” or “in an embodiment” in various places throughout this specification are not necessarily referring to the same embodiment of the invention.

The steps of the method described in connection with the aspects disclosed herein may be embodied directly in hardware, in a software module executed by a processor, or in a combination of the two. A software module (e.g., including executable instructions and related data) and other data may reside in a data memory such as RAM memory, flash memory, ROM memory, EPROM memory, EEPROM memory, registers, a hard disk, a removable disk, a CD-ROM, or any other form of computer-readable storage medium known in the art. A sample storage medium may be coupled to a machine such as, for example, a computer/processor (which may be referred to herein, for convenience, as a “processor”) such that the processor can read information (e.g., code) from and write information to the storage medium. A sample storage medium may be integral to the processor. The processor and the storage medium may reside in an ASIC. The ASIC may reside in user equipment. Alternatively, the processor and the storage medium may reside as discrete components in user equipment. Moreover, in some aspects any suitable computer-program product may comprise a computer-readable medium comprising codes relating to one or more of the aspects of the disclosure. In some aspects a computer program product may comprise packaging materials.

The above paragraphs describe many aspects. Obviously, the teaching of the invention can be accomplished by many methods, and any specific configurations or functions in the disclosed embodiments only present a representative condition. Those who are skilled in this technology will understand that all of the disclosed aspects in the invention can be applied independently or be incorporated.

While the invention has been described by way of example and in terms of preferred embodiment, it is to be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. A diagnostic assistance method, comprising:
generating, by a stethoscope, an audio data;
generating, by an ultrasonic device, an image data;
processing, by a first processing module, the audio data to generate a first result;
processing, by a second processing module, the image data to generate a second result
generating a diagnostic assistance result according to the first result and the second result.

2. The diagnostic assistance method of claim 1, wherein the first processing module processes the audio data through a first algorithm to generate the first result, and the second processing module processes the image data through a second algorithm to generate the second result.

3. The diagnostic assistance method of claim 2, wherein the first algorithm is a Recurrent Neural Network (RNN) deep learning algorithm and the second algorithm is a Convolutional Neural Network (CNN) deep learning algorithm.

4. The diagnostic assistance method of claim 2, further comprising:
comparing a training audio data with the audio data through the first algorithm to generate the first result; and
comparing a training image data with the image data through the second algorithm to generate the second result.

5. The diagnostic assistance method of claim 1, further comprising:
analyzing, by a third processing module, the first result and the second result using a third algorithm to generate the diagnostic assistance result.

6. The diagnostic assistance method of claim 5, wherein the third algorithm is an Ensemble Learning algorithm.

7. The diagnostic assistance method of claim 1, wherein the stethoscope is a digital stethoscope.

8. The diagnostic assistance method of claim 1, wherein the ultrasonic device is an ultrasonic probe.

9. The diagnostic assistance method of claim 1, further comprising:
displaying the diagnostic assistance result in a display device.

10. The diagnostic assistance method of claim 1, wherein the diagnostic assistance result is marked audio data, marked image data, or text data.
